(19) United States
(12) Patent Application Publication
(10) Pub. No.: US 2007/0244167 A1
(43) Pub. Date: Oct. 18, 2007

(54) Method for Purificaion of Amlodipine Free Base

(76) Inventors: Daniel Spielmann, Obergosgen (CH); Ralf Hoss, Zofingen (CH); Beat W. Muller, Therwil/BL (CH); Michael Oelting, Zofingen (CH)

Correspondence Address: Daniel A Scola;Hoffmann & Baron, 6900 Jericho Turnpike, Syosset, NY, 11791, US

(21) Appl. No.: 10/565,605

(22) PCT Filed: Jul. 19, 2004
(86) PCT No.: PCT/CH04/00457

§ 371 Date: Feb. 12, 2007

(30) Foreign Application Priority Data
Jul. 25, 2003 (CH) 1302/03

(51) Int. Cl. C07D 211/90; A61K 31/44

(52) U.S. Cl. 514/356; 546/321

(57) ABSTRACT

Summary A method for the purification of the deprotected free amlodipine base, present in the reaction mixture following deprotection of an N-protected amlodipine compound, whereby (i) the reaction mixture containing the deprotected amlodipine free base, optionally after a preceeding filtration, is evaporated to dryness, or the solvent removed from the reaction mixture, (ii) the amlodipine free base contained in the residue thus obtained, is dissolved in a suitable solvent and (iii) the amlodipine free base is precipitated from said solution.

The present invention relates to a method for the purification of the deprotected free amlodipine base. The free amlodipine base corresponds to the chemical formula (I) 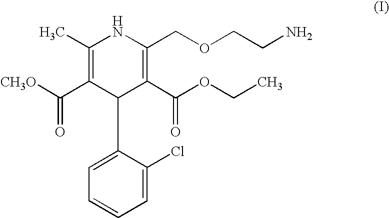
and is chemically known as 2-[2-(aminoethoxy)methyl]-4-(2-chlorophenyl)-3-ethoxycarbonyl-5-methoxycarbonyl-6-methyl-1,4-dihydropyridine. The free amlodipine base as well as the salts thereof are also known. These compounds are used as pharmaceutically active calcium antagonists with excellent antiischemic and antihypertensive effect.

In EP 0 089 167 the preparation of the free amlodipine base as well as the salts thereof are described. In EP 0 089 167, by way of an example, first a derivative of amlodipine is produced with the help of the “Hantzsch Reaction”, preferably phthalimidoamlodipine. From this, by hydrolysis the deprotected amlodipine base i.e. the free amlodipine base is obtained, and subsequently a salt e.g. amlodipine maleate is produced. The free amlodipine base is isolated from the reaction mixture by filtration to remove all possible residues and evaporation of the reaction solution filtrate for removing the solvent (ethanol). The free amlodipine base contained in the residue obtained after evaporation of the reaction solvent is dissolved in a suitable solvent and processed further to produce the salt.

In WO 02/053135 the deprotected amlodipine base is precipitated directly from the reaction mixture obtained after the hydrolysis, for instance by adding water or another compound, which causes the precipitation. The purpose of WO 02/053135 is to avoid or totally exclude the evaporation of the solvent as given in EP 0 089 167.

It has now been found that it is surprisingly advantageous to use the puress used in EP 0 089 167, for the isolation of the free deprotected amlodipine base and subsequently with the help of a suitable solvent dissolve the residue containing the free deprotected amlodipine base thus obtained to form a solution and then precipitate the free amlodipine base from the solution to purify it or crystallise it or use it in a subsequent process to make a salt. It was ascertained that the use of such a process does not in any way hamper the economic viability of the subsequent production of pure free amlodipine base which is obtained in high yield and purity.

The present invention has been defined in the patent claims. In particular, the present invention relates to a method for the purification of the deprotected free amlodipinee base, present in the reaction mixture following deprotection of an N-protected amlodipinee compound, whereby (i) the reaction mixture containing the deprotected amlodipinee free base optionally after a preceeding filtration, is evaporated to dryness, or the solvent removed from the reaction mixture, (ii) the amlodipinee free base, contained in the residue thus obtained, is dissolved in a suitable solvent and (iii) the amlodipinee free base is precipitated from said solution.

The amlodipinee base precipitated in this way can be purified further, if required, or crystallised or processed further, to make a salt.

Suitable salts of the amlodipine base are, for instance, the salts with known organic and inorganic acids, such as the hydrochloride, hydrobromide, sulphate, hydrogen sulphate, maleate, hydrogen maleate, fumarate, besylate, besylate monohydrate, mesylate, mesylate monohydrate, citrate or tartrate.

The reaction mixture with the deprotected free amlodipine base contains as solvent preferably the solvents specified in EP 0 089 167, preferably ethanol, whereby the method specified in EP 0 089 167 is used preferably for preparing the deprotected free amlodipine base. These solvents preferably have a low boiling point and can be easily removed from the reaction mixture, or the reaction mixture can be easily evaporated till it dries.

The free amlodipine base contained in the residue obtained after evaporation of the reaction solvent is dissolved in a suitable solvent, and if required, a higher temperature is used. Such solvents are for example aliphatic (C₄₋₁₈)-hydrocarbons, alcohols, polyols, low-molecular polyalkylene oxides, halogenated hydrocarbons, alkyl esters, linear and cyclic ethers, ketones, aprotic solvents and mixtures thereof. Preferred are the polar solvents.

Aliphatic (C₄₋₁₈)-hydrocarbons are, for instance ethane, propane, butane, hexane and the homologous hydrocarbons. Alcohols are preferably methanol or ethanol. Polyols preferably imply ethylene glycol. Low-molecular polyalkylene oxides preferably are diethylene glycol or triethylene glycol. Halogenated hydrocarbons are preferably dichloromethane, trichloromethane, dibromomethane and tribromomethane. Alkyl esters preferably are ethyl acetate. Linear and cyclic ethers are preferably diethylether, dipropylether, dioxane. Ketones preferably are dimethylketone. Aprotic solvents preferably are dimethylsulphoxide (DMSO) or dimethylformamide (DMF) or acetonitrile.

Preferred are the halogenated hydrocarbons, alkyl esters, linear and cyclic ethers, ketones, aprotic solvents and mixtures thereof.

For precipitating the free amlodipine base from the solution one adds a second solvent, herein referred to as counter-solvent, which has a different dipole moment to the first solvent. Such a counter solvent is, for instance, water, when the first solvent is miscible with water. Preferred are the aliphatic hydrocarbons, such as hexane, heptane or octane as counter solvents. The miscibility of such solvents and counter-solvents can easily be optimised by the persons skilled in the art with the help of tests. Thus, dissolving the residue in a solvent can be done if required at a higher temperature, a counter-solvent is added, and a precipitation is carried out at a lower temperature. Such methods are known to persons skilled in the art. The examples illustrate the invention.

### EXAMPLE 1

a) Phthalimidoamlodipine is treated in the presence of hydrazine hydrate in ethanol as per the example 22, method B of EP 0 089 167, till the free amlodipine base is released. The reaction mixture is filtered. The filtrate is thereafter evaporated in vacuum till it dries. The residue is then dissolved in methylene chloride as first solvent. Then, cyclohexane is added as the counter-solvent and cooled to a temperature of about 0-2° C., till the free base precipitates. This is then filtered out and dried. One obtains the free base in powder form.

b) One gets a similar result, when one dissolves in ethyl acetate or propyl acetate as the first solvent and adds adequate amounts of cyclohexane or heptane as counter solvent and cools to a temperature of about 0-2° C. when the free base precipitates.

1. A method for the purification of the deprotected free amlodipine base, present in the reaction mixture following deprotection of an N-protected amlodipine compound, whereby (i) the reaction mixture containing the deprotected amlodipine free base, optionally after a preceeding filtration, is evaporated to dryness, or the solvent removed from the reaction mixture, (ii) the amlodipine free base contained in the residue thus obtained, is dissolved in a suitable solvent and (iii) the amlodipine free base is precipitated from said solution.

2. A method as claimed in claim 1, further comprising isolating the precipitated amlodipine base and further purifying it and/or crystallizing it and/or processing it directly to make a salt.

3. A method as claimed in claim 2, characterised in that the amlodipine base is further processed to hydrochloride, hydrobromide, sulphate, hydrogen sulphate, maleate, hydrogen maleate, fumarate, besylate, besylate monohydrate, mesylate, mesylate monohydrate, citrate or tartrate.

4. A method as claimed in claim 1, wherein the solvent used in step (ii) is selected from the group comprising aliphatic (C₄₋₁₈) hydrocarbons, alcohols, polyols, low-molecular polyalkylene oxide, halogenated hydrocarbons, alkyl ester, linear and cyclic ethers, ketones, aprotic solvents and mixtures thereof, preferably a polar solvent.

5. A method as claimed in claim 4, wherein the solvent is selected from the group comprising ethane, propane, butane, hexane and the homologous hydrocarbons; methanol, ethanol; ethylene glycol; diethylene glycol, triethylene glycol; dichloromethane, trichloromethane, dibromomethane and tribromomethane; ethyl acetate; diethyl ether, dipropyl ether, dioxane; dimethyl ketone; dimethyl sulphoxide, dimethyl formamide and acetonitrile.

6. A method as claimed in claim 5, wherein the solvent is selected from the group comprising halogenated hydrocarbons, alkyl esters, linear or cyclic ethers, ketones, aprotic solvents and mixtures thereof.

7. A method as claimed in claim 1, wherein the precipitation in step (iii) is carried out with a second solvent or with a counter-solvent, which has a different dipole moment to the solvent used in step (ii).

8. A method as claimed in claim 7, wherein the counter-solvent is selected from amongst water and aliphatic hydrocarbons, such as hexane, heptane or octane.

9. A method as claimed in claim 8, wherein one uses an aprotic compound as the solvent in step (ii) and water as the counter-solvent in step (iii).
